(19) United States
(12) Patent Application Publication
(10) Pub. No.: US 2012/0203040 A1
(43) Pub. Date: Aug. 9, 2012

(54) Process for the Production of Paraffinic Hydrocarbons

(75) Inventor: Paul Ratnasamy, Pune (IN)

(73) Assignee: UNIVERSITY OF LOUISVILLE RESEARCH FOUNDATION, INC., Louisville, KY (US)

(21) Appl. No.: 13/365,789

(22) Filed: Feb. 3, 2012

Related U.S. Application Data
(60) Provisional application No. 61/439,112, filed on Feb. 3, 2011.

(51) Int. Cl. C07C 9/00; C07C 5/27; C07C 7/00

(52) U.S. Cl. 585/16; 585/733; 585/310

(57) ABSTRACT

Reaction catalysts and supports as disclosed herein are utilized in a process for converting carboxylic acids, which are derived from molecules found in various feedstocks of biological origin as well as various byproducts of industrial processes, to linear paraffinic hydrocarbons, the latter being capable of use in various applications, including as an alternative source of fuel.

### CROSS REFERENCE TO RELATED U.S. APPLICATION

This application claims priority to U.S. Provisional Application No. 61/439,112 filed on Feb. 3, 2011.

### FIELD OF INVENTION

The invention relates to the conversion of carboxylic acids obtained from biomass and other natural or industrial sources into paraffinic hydrocarbons suitable for use as fuels, or as starting materials for other derivatives and products.

### BACKGROUND

Hydrocarbons are an energy source for internal combustion engines, for turbines in jet aircraft, and for other kinds of engines, as well as for other applications that require a source of fuel. Some hydrocarbon fuels are linear, paraffinic hydrocarbons. Some, such as, for example, kerosene and some jet fuels, have 10-to-15 carbon atoms in their molecular structure. By comparison, diesel fuels typically have between 15-to-22 carbon atoms.

Hydrocarbon fuels and other petrochemical products are obtained from crude petroleum oil through a series of conventional steps. These include, but are not necessarily limited to, distillation followed by additional refining. Attempts are being made, however, to produce hydrocarbon fuels from alternative, renewable sources, including but not limited to feedstocks of biological origin. A common objective of such attempts has been to develop hydrocarbon fuels with similar chemical and functional properties to fuels that are obtained from crude petroleum, but from alternative sources and without having to utilize the conventional steps such as those mentioned above. Moreover, because of their similar chemical properties and functional properties, some hydrocarbon fuels from alternative, renewable sources are compatible with and, therefore, acceptable for use with, the kinds of engines for which petroleum-derived hydrocarbon fuels are intended.

More specifically, hydrocarbon fuels from alternative, renewable sources other than petroleum, include those products which are obtained from a process according to multiple embodiments and alternatives as described and claimed herein. In some cases, such products are capable of being stored and transported through existing infrastructure (e.g., storage tanks and pipelines) as with petroleum-derived hydrocarbon fuels. This increases the feasibility of using such products as replacements for petroleum-derived hydrocarbon fuels in their applications as transportation fuels, as well as other applications that require a source of energy.

Linear, paraffinic hydrocarbons do not occur naturally in large supply. However, one route for the production of these hydrocarbons is through decarboxylation of carboxylic acids, which are readily available in the lipid portion of biomass raw materials. The fact that biomass raw materials are in relatively large supply increases desirability of this approach. For example, the lipid portions of plant oils, animal fats, animal oils and algae oils are a ready source of triglyceride esters, which are converted to carboxylic acids through methods known to persons of ordinary skill in the art.

Through such methods, carboxylic acids are obtained from biomass raw materials, and are used as starting materials in the conversion to linear, paraffinic hydrocarbons. As a non-limiting example, stearic acid, with a chemical formula of C₁₇H₃₅COOH (or, C₁₈H₃₆O₂), is a common carboxylic acid derived from biomass raw materials. Various routes exist for the conversion of stearic acid to a linear, paraffinic hydrocarbon, which require the removal of the two oxygen atoms of the carboxylate group. One approach previously studied and reported on, known as hydrodeoxygenation, accomplishes this but requires the use of hydrogen as a starting material in the reaction. However, an alternative approach is decarboxylation, which involves removal of the carboxylate group, as carbon dioxide. In this approach, the alkane product has one carbon atom less than the carboxylic acid starting material. This decarboxylation reaction does not require a supply of hydrogen as a reactant.

Accordingly, decarboxylation produces hydrocarbons with a linear structure in which the alkyl group of the carboxylic acid is preserved, and the carboxylate group (i.e., one carbon atom and two oxygen atoms) is removed as carbon dioxide. Decarboxylation of carboxylic acids is generally less expensive today than the hydrodeoxygenation approach, in which the manufacturer must also obtain a supply of hydrogen as a reactant.

Also, decarboxylation yields normal, linear, paraffinic hydrocarbons, without having to use hydrogen as a reactant. These products are then isolated and separated by fractional distillation or other methods known to persons of ordinary skill in the art, into appropriate fuel fractions for use as kerosene, jet fuel, diesel fuel, automobile fuel or other kinds of fuel as selectably desired by an end user.

### SUMMARY OF INVENTION

A process for producing linear, paraffinic hydrocarbons converts fatty acids (a.k.a. carboxylic acids) containing 6-to-24 carbon atoms into linear, paraffinic hydrocarbons, which can be used as fuels and for other applications that require a source of energy. Generally, the linear, paraffinic hydrocarbons contain one less carbon atom than the starting material. As a non-limiting example, decarboxylation of stearic acid (18 carbons) produces heptadecane (17 carbons, C₁₇H₃₆).

In some embodiments, surface basicity of the catalyst and its dispersibility over the support are factors for selecting a reaction catalyst. Porosity and/or mesoporosity, as well hydrophobicity, are factors for selecting a support.

The present process produces linear, paraffinic hydrocarbons which can be used in various ways. In some applications, these products are used as hydrocarbon fuels. Alternatively, these products are starting materials, which are converted to branched-chain paraffinic hydrocarbons through processes known to persons of ordinary skill in the art, and which are suitable for use as hydrocarbon fuels. Alternatively, these products are starting materials for the production of various petrochemicals, through methods which are known to persons of ordinary skill in the art. Non-limiting examples of such petrochemicals include linear alpha olefins, alpha olefin sulfonates, and linear alkyl benzenes. Such petrochemicals are used in the manufacture of various end products. For example, linear alkyl benzenes are used in the manufacture of detergents. Other examples of petrochemicals used in the manufacture of various end products include high viscosity index star polymers. The forgoing are non-limiting examples of a broad scope in which products of the subject process are used.

### MULTIPLE EMBODIMENTS AND ALTERNATIVES

A process for producing linear, paraffinic hydrocarbons comprises (1) obtaining a supply of at least one carboxylic acid; (2) selecting a reaction catalyst and a support as described herein; and (3) contacting the at least one carboxylic acid with the catalyst over the support, under conditions as described herein, resulting in the decarboxylation reaction:

R—COOH→R—H+CO₂.

In some embodiments, linear, paraffinic hydrocarbons are then isolated from the end products of the reaction. The linear, paraffinic hydrocarbons obtained as the end products of the decarboxylation reaction are fully saturated hydrocarbons, which are appropriate to be used in the various applications described above.

In some embodiments, the at least one carboxylic acid is a carboxylic acid having 6-to-24 carbon atoms. Optionally, the at least one carboxylic acid is a mixture of at least two carboxylic acids, each having 6-to-24 carbon atoms. In some embodiments, triglyceride esters contained in various sources as described below are converted to carboxylic acids through methods known to persons of ordinary skill in the art. In some embodiments, the at least one carboxylic acid is obtained from a renewable feedstock of biological origin (i.e., biomass raw materials), such as, for example, plant oils, animal fats, animal oils, and algae oils. Optionally, the source of the at least one carboxylic acid consists of a mixture of two or more members of this group. Alternatively, the at least one carboxylic acid is obtained from an industrial or other non-biological source, such as, for example industrial greases, and waxes obtained from solid wastes, and paper mills.

In some embodiments, starting materials used in a process for producing linear, paraffinic hydrocarbons are saturated carboxylic acids. Alternatively, the starting materials are unsaturated carboxylic acids. In the latter case, it is an option to reduce the unsaturated carboxylic acids to saturated carboxylic acids by reaction with hydrogen, through methods known to persons of ordinary skill in the art, before undergoing decarboxylation.

The decarboxylation reaction (also referred to herein as, “decarboxylation”) by which carboxylic acids are converted to linear, paraffinic hydrocarbons, is exothermic. Notwithstanding its favorable thermodynamics, however, the energy associated with the transition state of this reaction represents an activation barrier that requires a catalyst in order for the reaction to proceed under the kinds of conditions disclosed herein. Therefore, in some embodiments, a catalyst is chosen from the group platinum, palladium, nickel, nickel-molybdenum, nickel-tungsten, and platinum-copper. In some embodiments, surface basicity is considered in selecting a catalyst. It will be noted that the presence or absence of various functional groups at the surface of a catalyst influences its surface basicity. In some embodiments, the surface basicity of the catalyst is determined by measuring the amount of acetic acid adsorbed from a 0.1 N solution of acetic acid in normal hexane, at room temperature, on a sample of the solid catalyst treated previously at high temperatures to remove impurities (e.g., water, carbon dioxide), and is expressed as equivalents of acetic acid adsorbed by the solid. Also, in general, the extent to which the metal catalyst is dispersed over the support influences the yield of the decarboxylation reaction. Stated differently, the hydrocarbon product yield increases as the level of metal dispersion increases. Preferably, the dispersion of the catalyst over the support is at least about 50%.

In some embodiments, a support is selected which is a porous or mesoporous structure formed from basic oxide materials, which are chosen from the group hydrotalcite, magnesium oxide, calcium oxide, a mixed oxide of ceria-zirconia, and lanthanum oxide. In some embodiments, hydrophobicity is considered in selecting a support for the catalyst. Hydrophobicity of the support is determined by measuring adsorption of water vapor by the support under ambient conditions. Preferably, the support adsorbs no more than about 0.5% wt water vapor under ambient conditions. Optionally, hydrophobicity is determined by exposing the support to water vapor at 25° C. and corresponding equilibrium pressures, and measuring the percentage of water vapor adsorbed by the support in relation to the weight of the catalyst to be used.

In some embodiments, carboxylic acid starting materials are diluted in a suitable solvent before commencing the decarboxylation reaction. As used herein, a suitable solvent would include hydrocarbons, such as, for example dodecane or hexadecane. In some embodiments, a mixture of two or more hydrocarbons is used as a solvent. In some embodiments, decarboxylation is carried out in a suitable solvent. Alternatively, decarboxylation is carried out in a solvent-free reaction chamber.

If desired, a process for production of paraffinic hydrocarbons, as described herein according to multiple embodiments and alternatives, is carried out in a reactor. In some embodiments, the reactor is a batch reactor. Alternatively, the reactor is a semi-batch reactor. Alternatively, the reactor is a continuous flow reactor. Optionally, the carboxylic acid starting materials are passed over a supported catalyst in a reaction zone contained within the reactor. In some embodiments, decarboxylation is carried out at a temperature in a range of 200° C.-400° C. Alternatively, the temperature is in a range of of 250° C.-350° C. In some embodiments, decarboxylation is carried out at a pressure in a range of 1 bar-60 bar.

In some embodiments, decarboxylation produces reaction products consisting of linear, paraffinic hydrocarbons, which are isolated and separated using techniques known to persons of ordinary skill in the art (e.g., distillation). In this way, the separated reaction products can be put to use according to their intended purpose as selected by a user.

Optionally, a process for producing linear hydrocarbons is used for the conversion of unsaturated carboxylic acids to olefinic (unsaturated), linear hydrocarbons. This alternative embodiments comprises (1) obtaining a supply of at least one carboxylic acid; (2) selecting a reaction catalyst and a support as described herein; and (3) contacting the at least one carboxylic acid with the catalyst over the support, under conditions as described herein, resulting in the decarboxylation reaction:

R—COOH→R—H+CO₂.

wherein R contains six-to-twenty-four carbons, and has at least one carbon-carbon double bond. Further, in some embodiments, both paraffinic and olefinic carboxylic acids are together converted to paraffinic and olefinic linear hydrocarbons in a single reaction chamber, using a catalyst, support, and reaction conditions according to alternative embodiments as set forth herein. Moreover, in some alternative embodiments, the process converts at least a portion of olefinic carboxylic acids to linear, paraffinic hydrocarbons.

Examples 1, 3, 4, 5, 6, 7, 10, 11, 12 and 13, which are described below, are alternative embodiments of a process for producing linear, paraffinic hydrocarbons. Examples 2, 8, and 9 are offered as comparative examples. Example 14 illustrates one application for the products of the subject process, namely as starting materials for conversion of paraffinic hydrocarbons to aviation turbine fuel (i.e., jet fuel).

In the examples, the catalytic run was carried out continuously over 150 hours. The liquid products contained two layers: a hydrocarbon top layer and a bottom, water layer, which were condensed and collected in a product receiver at the end of 50 and 150 hours. The identity of the hydrocarbon products was determined by gas chromatography using a Hewlett Packard 4890 gas chromatograph. The acid number of the products of the hydrocarbon layer were determined, and compared to the acid number of the carboxylic acid feedstock. Based upon that comparison, the conversion of the carboxylic acid feedstock to paraffinic hydrocarbons was determined, as further described below. Iodine number is an indicator of unsaturation and presence of carbon-carbon double bonds.

### EXAMPLE 1

### A Palladium Metal Catalyst Supported on a Basic Hydrotalcite Oxide Support

The surface area of the support was approximately 99.5 m² per gram; the pore volume of the support was approximately 0.3 ml per gram of catalyst. The support was dried overnight at 150° C. The average pore width of the catalyst was approximately 9.5 nm (nanometers); basicity of the catalyst, as determined by titration with 0.1 normal acetic acid, was approximately 0.15 mEq (milliequivalent) acetic acid per gram.

An aqueous solution of palladium nitrate containing a sufficient amount of palladium metal for 5 wt % of palladium in the final catalyst was prepared. A suspension of the hydrotalcite support in the palladium nitrate solution was prepared. A 0.1 normal aqueous solution of sodium hydroxide, of volume at least equal to the pore volume of the catalyst, was added to that suspension. The palladium compound was reduced to the metallic state by addition of a sufficient amount of sodium borohydride. The catalyst was dried in a flow of nitrogen at 200° C. for 5 hours.

The reduced catalyst was loaded in a downflow, fixed bed reactor. A feedstock consisting of a mixture of oleic acid and normal hexadecane in equal weight proportions was passed over the catalyst with a HPLC pump at a temperature of 330° C., hydrogen pressure of 20 bar, a hydrogen to oleic acid ratio of 600 (V/V) and a weight hourly space velocity of oleic acid of 1.0. The gaseous product of the reaction was carbon dioxide.

In the liquid phase, the conversion of the oleic acid to hydrocarbon products was 94 wt % after 50 hours and 92 wt % after 150 hours. The hydrocarbon layer contained penta-, hexa, hepta- and octa-decanes. The iodine number of these products was negligible, indicating that the product contained only saturated paraffinic hydrocarbons. From the ratio of heptadecane to (heptadecane+octadecane), i.e., C17/(C17+C18)), the selectivity for decarboxylation was calculated to be 92% after 50 hours and 94% after 150 hours. The catalyst was evaluated according to conventional methods and determined not to have been deactivated.

### EXAMPLE 2

### A Palladium Metal Catalyst Supported on a Non-Basic Support Material, i.e., Activated Carbon

The surface area of the support was approximately 778 m² per gram; the pore volume of the support was approximately 0.45 ml per gram of catalyst. The support was dried overnight at 150° C. The average pore width of the catalyst was approximately 3.3 nm; basicity of the catalyst, as determined by titration with 0.1 normal acetic acid, was approximately 0.02 mEq acetic acid per gram.

An aqueous solution of palladium chloride containing a sufficient amount of palladium metal for 5 wt % of palladium in the final catalyst was prepared and deposited on the carbon by incipient deposition. The catalyst was dried in a flow of nitrogen at 200° C. for 5 hours. The dried catalyst was loaded in a downflow, fixed bed reactor and reduced in flowing hydrogen at 250° C., 20 bar pressure for 6 hours. A feedstock consisting of a mixture of oleic acid and normal hexadecane in equal weight proportions was passed over the catalyst with a HPLC pump at a temperature of 330° C., hydrogen pressure of 20 bar, a hydrogen to oleic acid ratio of 600(V/V) and a weight hourly space velocity of oleic acid of 1.0. The gaseous product of the reaction was carbon dioxide.

In the liquid phase, the conversion of the oleic acid to hydrocarbon products was 98 wt % at the end of 50 hours and 69 wt % after 150 hours. The hydrocarbon layer contained penta-, hexa-, hepta- and octa decanes. The iodine number of these products was negligible, indicating that the product contained only saturated paraffinic hydrocarbons. From the ratio of heptadecane (C17) to octadecane (C18), the selectivity for decarboxylation was calculated to be only 43% after 50 hours. Thus, even though this catalyst was active for the deoxygenation reaction, it deactivated at the end of 150 hours and most of the oxygen of the carboxyl group was removed as H₂O rather than as CO₂. As a consequence, hydrogen consumption during the process was relatively high.

### EXAMPLE 3

### A Palladium Metal Catalyst Supported on a Basic Hydrotalcite Oxide Support; Reaction not Carried Out Under Hydrogen Pressure

Process conditions were the same as for Example 1, except that the reaction was not carried out under hydrogen; the pressure within the reactor remained at 20 bar of nitrogen throughout the reaction. The gaseous product of the reaction was carbon dioxide.

The hydrocarbon layer contained penta-, hexa-, hepta- and octa decanes. The iodine number of these products was negligible, indicating that the product contained only saturated paraffinic hydrocarbons. The conversion of the oleic acid to hydrocarbon products was 94 wt % at the end of 50 hours. From the ratio of heptadecane to (heptadecane+octadecane), the selectivity for decarboxylation was calculated to be 93%.

### EXAMPLE 4

### A Palladium Metal Catalyst Supported on a Magnesium Oxide Support

The surface area of the support was approximately 99.5 m² per gram; the pore volume of the support was approximately 0.23 ml per gram of catalyst. The support was dried overnight at 250° C. The average pore width of the catalyst was approximately 3.4 nm; basicity of the catalyst, as determined by titration with 0.1 normal acetic acid, was approximately 0.11 mEq acetic acid per gram.

An aqueous solution of palladium nitrate containing a sufficient amount of palladium metal for 5 wt % of palladium in the final catalyst was prepared. A suspension of the magnesium oxide support in the palladium nitrate solution was prepared. A 0.1 normal aqueous solution of sodium hydroxide, of volume at least equal to the pore volume of the solid, was added to that suspension. The palladium compound was reduced to the metallic state by addition of a sufficient amount of sodium borohydride. The catalyst was dried in a flow of nitrogen at 200° C. for 5 hours.

The reduced catalyst was loaded in a downflow, fixed bed reactor. A feedstock consisting of a mixture of oleic acid and normal hexadecane in equal weight proportions was passed over the catalyst with a HPLC pump at a temperature of 330° C., hydrogen pressure of 30 bar, a hydrogen to oleic acid ratio of 600 (V/V) and a weight hourly space velocity of oleic acid of 1.0. The gaseous product of the reaction was carbon dioxide.

In the liquid phase, the conversion of the oleic acid to hydrocarbon products was 95 wt % at the end of 50 hours and 94 wt % after 150 hours. The hydrocarbon layer contained penta-, hexa-, hepta- and octa-decanes. From the ratio of heptadecane to (heptadecane+octadecane), the selectivity for decarboxylation was calculated to be 86% after 50 hours and 90% after 150 hours. The catalyst was evaluated according to conventional methods and determined not to have been deactivated.

### EXAMPLE 5

### A Palladium Metal Catalyst Supported On A Basic Hydrotalcite Oxide Support; Reaction Carried Out Under Nitrogen Pressure

Process conditions were the same as for Example 1, except that the reaction was carried out under nitrogen; the pressure within the reactor remained at 20 bar of nitrogen throughout the reaction. The gaseous product of the reaction was carbon dioxide. In the liquid phase, the conversion of oleic acid was 97 wt %, and its selectivity (C17/(C17+C18)) value was 91%.

### EXAMPLE 6

### A Nickel Metal Catalyst Supported on a Mixed Oxide Support of Ceria-Zirconia

A ceria-zirconia mixed oxide support was prepared by coprecipitation of the mixed hydroxides of cerium and zirconium from an aqueous solution of the nitrates using sodium hydroxide as the precipitating agent. The support had a surface area of approximately 163 m² per gram; the pore volume of the support was approximately 0.165 ml per gram of catalyst.

The support was then impregnated with a nickel nitrate solution by the incipient wetness method to yield 41.54 wt % of nickel oxide in the final catalyst. The material was dried in air at 120° C. for 12 hours and calcined in air at 400° C. for 12 hours. Basicity of this catalyst, as determined by titration with 0.1 normal acetic acid, was approximately 0.21 mEq acetic acid per gram. The contents of cerium and zirconium oxides in the final catalyst were 25.6 wt % and 32.8 wt %, respectively. Surface area of the final catalyst was 134 m² per gram, and its pore volume was 0.12 ml per gram.

Except as stated above, process conditions were the same as for Example 1. In the liquid phase, the conversion of the oleic acid to hydrocarbon products was 85 wt % at the end of 50 hours and 81 wt % after 150 hours. The selectivity (C17/(C17+C18)) value was 84% after 50 hours and 89% after 150 hours.

### EXAMPLE 7

### A Palladium Metal Catalyst Supported on Basic Hydrotalcite Catalyst as in Example 1, From a Mixture of Dodecanoic Acid and Oleic Acid

This example used the catalyst and support of Example 1 with the same process conditions as Example 1, except as noted with respect to temperature and pressure. An equimolar mixture of dodecanoic acid (C₁₂H₂₄O₂) and oleic acid (C₁₈H₃₄O₂)—with a weight hourly space velocity of the combined fatty acids of 1.0—underwent decarboxylation in hexadecane solvent, at 295° C., and a hydrogen pressure of 10 bar. The conversion of dodecanoic acid (as determined by gas chromatography) was 90%, and the conversion of oleic acid (as determined by gas chromatography) was 96%. The selectivity in the conversion of dodecanoic acid (C11/(C11+C12)) value was 86%, and the corresponding value for the selectivity in the conversion of oleic acid (C17/(C17+C18) was 91%.

### EXAMPLE 8

### A Palladium Metal Catalyst Supported on a Non-Basic Support Material, i.e., Activated Acidic Aluminum Oxide

The surface area of the support was approximately 178 m² per gram; the pore volume of the support was approximately 0.35 ml per gram of catalyst. The support was dried overnight at 150° C.

The average pore width of the catalyst was approximately 2.3 nm; basicity of the catalyst, as determined by titration with 0.1 normal acetic acid, was approximately 0.03 mEq acetic acid per gram. An aqueous solution of palladium chloride containing a sufficient amount of palladium metal for 5 wt % of palladium in the final catalyst was prepared and deposited on the support by incipient deposition. The catalyst was dried in a flow of nitrogen at 200° C. for 5 hours.

The dried catalyst was loaded in a downflow, fixed bed reactor and the palladium metal was reduced in flowing hydrogen at 350° C., 20 bar pressure for 6 hours. A feedstock consisting of a mixture of oleic acid and normal hexadecane in equal weight proportions was passed over the catalyst with a HPLC pump at a temperature of 350° C., hydrogen pressure of 30 bar, a hydrogen to oleic acid ratio of 600 (V/V) and a weight hourly space velocity of oleic acid of 1.0. The gaseous products of the reaction were carbon dioxide, ethane, propane, butane, as well as propylene and butane.

In the liquid phase, the conversion of the palmitic acid to hydrocarbon products was 78 wt % at the end of 50 hours and 39 wt % after 150 hours. The hydrocarbon layer contained penta-, hexa-, hepta- and octa-decanes as well as olefins. The iodine number of the product, an indicator of unsaturation and presence of carbon-carbon double bonds, was 35 indicating that the product contained some olefins in addition to the saturated paraffinic hydrocarbons. From the ratio of heptadecane to octadecane, the selectivity for decarboxylation was calculated as 42% at the end of 50 hours. The catalyst was evaluated according to conventional methods, and was found to have sustained severe catalytic deactivation. Thus, even though this catalyst was active for the deoxygenation reaction, it deactivated fast and most of the oxygen of the carboxylate group was removed as H₂O rather than as CO₂. As a consequence, hydrogen consumption during the process was relatively high.

### EXAMPLE 9

### A Nickel Oxide-Molybdenum Oxide Catalyst Supported on a Non-Basic Support Material

The surface area of the support was approximately 212 m² per gram; the pore volume of the support was approximately 0.23 ml per gram of catalyst. The catalyst was prepared by the deposition of ammonium molybdate and nickel nitrate on aluminium oxide, drying at 120° C. and calcining it further in air at 500° C. The average pore width of the catalyst was approximately 2.5 nm; basicity of the catalyst, as determined by titration with 0.1 normal acetic acid, was approximately 0.03 mEq acetic acid per gram.

The catalyst was loaded in a downflow, fixed bed reactor and dried overnight at 150° C. to remove adsorbed matter like water and carbon dioxide. The catalyst was then sulfided for 24 hours at 200° C. in a stream of hexadecane containing 100 ppm of dimethyl disulfide.

A feedstock consisting of a mixture of oleic acid and normal hexadecane in equal weight proportions was then passed over the catalyst with a HPLC pump at a temperature of 330° C., hydrogen pressure of 45 bar, a hydrogen to oleic acid ratio of 1200 (V/V) and a weight hourly space velocity of oleic acid of 1.5. The gaseous product of the reaction was carbon dioxide.

In the liquid phase, the conversion of the oleic acid to hydrocarbon products was 90 wt % at the end of 50 hours and 85 wt % after 150 hours. The hydrocarbon layer contained penta-, hexa-, hepta- and octa-decanes. The iodine number of these products, indicating that the product contained only saturated paraffinic hydrocarbons and no olefins. From the ratio of heptadecane to octadecane, the selectivity for decarboxylation was calculated to be 60%.

These results indicated that even though the catalyst was active for the deoxygenation reaction over 150 hours, most of the oxygen of the carboxyl group was removed as H₂O rather than as CO₂. As a consequence, its selectivity for decarboxylation was relatively low, and hydrogen consumption during the process was relatively high.

### EXAMPLE 10

### A Palladium Metal Catalyst Supported on a Basic Calcium Oxide Support

The support was prepared by the decomposition of calcium carbonate at 650° C. in air. The surface area of the support was approximately 76.8 m² per gram; the pore volume of the support was approximately 0.28 ml per gram of catalyst. The support was dried overnight at 200° C. The average pore width of the catalyst was approximately 2.5 nm; basicity of the catalyst, as determined by titration with 0.1 normal acetic acid, was approximately 0.23 mEq acetic acid per gram.

An aqueous solution of palladium nitrate containing a sufficient amount of palladium metal for 5 wt % of palladium in the final catalyst was prepared. A suspension of the calcium oxide support in the palladium solution was prepared. The palladium compound was reduced to the metallic state by addition of a sufficient amount of sodium borohydride. The catalyst was dried in a flow of nitrogen at 200° C. for 5 hours.

The reduced catalyst was loaded in a downflow, fixed bed reactor. A feedstock consisting of a mixture of palmitic acid and normal dodecane in equal weight proportions was passed over the catalyst with a HPLC pump at a temperature of 335° C., hydrogen pressure of 20 bar, a hydrogen to palmitic acid ratio of 600 (V/V) and a weight hourly space velocity of palmitic acid of 1.0. The gaseous product of the reaction was carbon dioxide.

In the liquid phase, the conversion of the palmitic acid to hydrocarbon products was 88 wt % at the end of 50 hours and 85 wt % after 150 hours. The hydrocarbon layer contained dodecane, penta- and hexa decanes. The iodine number of these products was negligible, indicating that the product contained mainly saturated paraffinic hydrocarbons. From the ratio of pentadecane to (pentadecane+hexadecane), the selectivity for decarboxylation was calculated to be 93% after 50 hours and 95% after 150 hours.

### EXAMPLE 11

### A Palladium Metal Catalyst Supported on a Basic Lanthanum Oxide Support

The support was prepared by the decomposition of lanthanum carbonate at 700° C. in air. The surface area of the support was approximately 85.7 m² per gram; the pore volume of the support was approximately 0.19 ml per gram of catalyst. The support was dried overnight at 300° C. The average pore width of the catalyst was approximately 3.6 nm; basicity of the catalyst, as determined by titration with 0.1 normal acetic acid, was approximately 0.21 mEq acetic acid per gram.

An aqueous solution of palladium nitrate containing a sufficient amount of palladium metal for 5 wt % of palladium in the final catalyst was prepared. A suspension of the lanthanum oxide support in the palladium nitrate solution was prepared. The palladium compound was reduced to the metallic state by addition of sufficient amount of sodium borohydride. The catalyst was dried in a flow of nitrogen at 200° C. for 5 hours.

The reduced catalyst was loaded in a downflow, fixed bed reactor. A feedstock consisting of a mixture of palmitic acid and normal dodecane in equal weight proportions was passed over the catalyst with a HPLC pump at a temperature of 335° C., hydrogen pressure of 20 bar, a hydrogen to palmitic acid ratio of 600 (V/V) and a weight hourly space velocity of palmitic acid of 1.0. The gaseous product of the reaction was carbon dioxide.

In the liquid phase, the conversion of the palmitic acid to hydrocarbon products was 90 wt % at the end of 50 hours and 85 wt % after 150 hours. The hydrocarbon layer contained dodecane, penta- and hexa-decanes. The iodine number of these products was negligible, indicating that the product contained mainly saturated paraffinic hydrocarbons. From the ratio of pentadecane to (pentadecane+hexadecane), the selectivity for decarboxylation was calculated to be 85% after 50 hours and 87% after 150 hours. The catalyst was evaluated according to conventional methods and determined not to have been deactivated.

### EXAMPLE 12

### A Palladium Metal Catalyst Supported on Basic Hydrotalcite Catalyst—Fatty Acids Obtained From Beef Tallow

The catalyst and support of Example 1 were used, along with the process conditions of Example 1, except as noted with respect to temperature and pressure. Feedstock was a 50:50 wt. % mixture of n-hexadecane and C14-C18 fatty acids, the latter being obtained by hydrolyzing beef tallow supplied by Emery Oleochemicals LLC, Cincinnati, Ohio. The beef tallow was black in color, with a density of 0.86 g/cc; flash point of 185° C.; acid number of 198.5; iodine number of 56.9. Fatty acid content of feedstock was 3% C14:0, 22% C16:0, 4% C16:1, 1% C17:0, 20% C18:0, 45% C18:1, and 5% C18:2, wherein “0” represents the saturated hydrocarbon, “1” represents a monounsaturated hydrocarbon, and “2” represents two C═C double bonds in the molecule.

80 gm of feedstock was heated in an autoclave with 4 gm of the catalyst of Example 1 at 350° C. and 30 bar pressure of nitrogen for 3 hrs. The yield of liquid product was 81 wt %, with the gaseous product being mainly carbon dioxide with 1-3% of methane. The product was light orange in color. The liquid product's acid number was 1.5, and its iodine number was 45. The freezing point of the liquid product was 16° C. Based upon the reduction of acid number, decarboxylation of fatty acids in the beef tallow to linear paraffin hydrocarbons was calculated at greater than 99 wt % after 3 hours. The hydrocarbon layer contained C8 to C18 saturated and unsaturated hydrocarbons. The hydrocarbon chain length distribution (in wt %) in the liquid product obtained by gas chromatography was as follows: 9.3% C8, 10.5% C9, 11.9% C10, 11.8% C11, 9.3% C12, 8.1% C13, 5.8% C14, 5.9$ C15, 17.3% C16, 4.1% C17, 3.5% C18, and 2.4% C19.

### EXAMPLE 13

### Palladium Catalyst Supported on Basic Lanthanum Oxide Support—Fatty Acids Obtained From Beef Tallow

80 gm of feedstock of Example 12 (mixture of fatty acids from beef tallow and n-hexadecane) was reacted with catalyst of Example 11 over support of Example 11, by heating in an autoclave with 4 gm of the aforementioned palladium metal catalyst supported on a basic lanthanum oxide support catalyst at 350° C. and 40 bar pressure of nitrogen for 3 hrs. The yield of liquid product was 78 wt %, with the gaseous product being mainly carbon dioxide with 2% of methane. The product was colorless. The acid number of the liquid product was 1.1 and its iodine number was 1.7. The freezing point of the liquid product was 17° C. Based upon reduction of acid number, decarboxylation of fatty acids in beef tallow to hydrocarbon products was calculated at greater than 99 wt % after 3 hours. The hydrocarbon layer contained C10 to C18 saturated and unsaturated hydrocarbons. The hydrocarbon chain length distribution according to gas chromatography (in wt %) of the liquid product obtained was 1.3% C10, 1.7% C11, 2.1% C12, 4.4% C13, 6.5% C14, 18.7% C15, 19.4% C16, and 45.6% C17.

### EXAMPLE 14

### Production of Aviation Turbine Fuel

The liquid product of Example 13 was reacted with a hydroisomerisation catalyst known in the art at 350° C. and a hydrogen pressure of 40 bars for 3 hrs. The product had a freezing point of −64 C, a total sulfur content of 4 ppm (per ASTM D-1266), a smoke point of 26.7 mm (ASTM D-1322), net heat of combustion of 43.8 MJ/Kg (ASTM D-4809); approximately 10% of the products boiled at a temperature of less than about 254° C. and about 90% boiled in a range between about 254° C.-300° C.; and the elemental composition by %wt of 85% carbon, 15% hydrogen, and 0% oxygen. The liquid sample obtained by hydroisomerisation of linear paraffins obtained in Example 13 meets ASTM D-1655, the standard specification for jet fuel.

It is to be understood that the embodiments described herein are not limited in their application to the details of the teachings and descriptions set forth herein, or as illustrated in the above examples. Rather, it will be understood that a process for the production of linear, paraffinic hydrocarbons, as described and claimed according to multiple embodiments disclosed herein, is capable of other embodiments and of being practiced or of being carried out in various ways.

Also, it is to be understood that the phraseology and terminology used herein is for the purpose of description and should not be regarded as limiting. The use herein of “including,” “comprising,” “e.g.,” “such as, for example,” “containing,” or “having” and variations of those words is meant in a non-limiting way to encompass the items listed thereafter, and equivalents of those, as well as additional items.

Accordingly, the foregoing descriptions are meant to illustrate a number of embodiments and alternatives, rather than to serve as limits on the scope of what has been disclosed herein. The descriptions herein are not intended to be exhaustive, nor are they meant to limit the understanding of the embodiments to the precise forms disclosed. It will be understood by those having ordinary skill in the art that modifications and variations of these embodiments are reasonably possible in light of the above teachings and descriptions.

I claim:

1. A process for converting fatty acids to linear, paraffinic hydrocarbons, comprising the steps of:
(a) contacting carboxylic acid reactants with a catalyst over a support; and
(b) isolating linear, paraffinic hydrocarbon products from other reaction products;
wherein the carboxylic acid reactants have a chemical formula represented by R—COOH, R has at least six and no more than twenty-four carbon atoms, the basicity of the catalyst is at least approximately 0.1 mEq, and the support is chosen from the group hydrotalcite, magnesium oxide, calcium oxide, a mixed oxide of ceria-zirconia, and lanthanum oxide.

2. The process of claim 1, wherein the catalyst is chosen from the group platinum, palladium, nickel, nickel-molybdenum, nickel-tungsten, and platinum-copper, and wherein the basicity of the catalyst is between about 0.11 mEq-0.23 mEq.

3. The process of claim 1, wherein the step of contacting carboxylic acid reactants with a catalyst over a support is carried out at a temperature of no greater than about 400° C.

4. The process of claim 3, wherein the step of contacting carboxylic acid reactants with a catalyst over a support is carried out at a temperature of between about 250° C.-350° C.

5. The process of claim 3, wherein carboxylic acid reactants are derived from a feedstock, which is chosen from the group plant oils, animal fats, animal oils, and algae oils.

6. The process of claim 2, wherein the support adsorbs no more than about 0.5% wt water vapor under ambient conditions.

7. The process of claim 2, wherein the dispersion of the catalyst over the support is at least about 50%.

8. A process for producing branched paraffinic hydrocarbons from fatty acids comprising the steps of claim 1, and further comprising a second reaction step of contacting linear, paraffinic hydrocarbon products produced with a hydroisomerisation catalyst to form at least one branched hydrocarbon.

9. The process of claim 8, wherein the branched paraffinic hydrocarbons have a boiling point between about 150°-300° C.

10. A paraffinic hydrocarbon produced by:
(a) contacting carboxylic acid reactants with a catalyst over a support; and
(b) isolating hydrocarbon products from other reaction products;
wherein at least one hydrocarbon product is a linear, paraffinic hydrocarbon, and
wherein the carboxylic acid reactants have a chemical formula represented by R—COOH, R has at least six and no more than twenty-four carbon atoms, the basicity of the catalyst is at least approximately 0.1 mEq, and the support is chosen from the group hydrotalcite, magnesium oxide, calcium oxide, a mixed oxide of ceria-zirconia, and lanthanum oxide.

11. The hydrocarbon of claim 10, wherein the catalyst is chosen from the group platinum, palladium, nickel, nickel-molybdenum, nickel-tungsten, and platinum-copper, and wherein the basicity of the catalyst is between about 0.11 mEq-0.23 mEq.

12. The hydrocarbon of claim 10, wherein the step of contacting carboxylic acid reactants with a catalyst over a support is carried out at a temperature of no greater than about 400° C.

13. The hydrocarbon of claim 12, wherein the step of contacting carboxylic acid reactants with a catalyst over a support is carried out at a temperature of between about 250° C.-350° C.

14. The hydrocarbon of claim 12, wherein carboxylic acid reactants are derived from triglycerides contained in a feedstock, wherein the feedstock is chosen from the group plant oils, animal fats, animal oils, and algae oils.

15. The hydrocarbon of claim 11, wherein the support adsorbs no more than about 0.5% wt water vapor under ambient conditions.

16. The hydrocarbon of claim 11, wherein the dispersion of the catalyst over the support is at least 50%.
